(19) United States
(12) Patent Application Publication
(10) Pub. No.: US 2004/0265990 A1
(43) Pub. Date: Dec. 30, 2004

(54) Biological compositions for reduction of E. coli infections

(76) Inventor: Ling Yuk Cheung, Hong Kong (HK)

Correspondence Address: JONES DAY, 222 EAST 41ST ST, NEW YORK, NY, 10017, US

(21) Appl. No.: 10/610,578

(22) Filed: Jun. 30, 2003

(51) Int. Cl. C12N 13/00; C12N 1/14; C12N 1/16

(52) U.S. Cl. 435/254.21; 435/254.2; 435/173.1

(57) ABSTRACT

The invention relates to biological compositions that can be used to prevent the spread of pathogenic Escherichia coli infections in the environment. The biological compositions of the invention comprise a plurality of live yeast cells which are capable of limiting or suppressing the growth of pathogenic E. coli O157:H7. The biological compositions can be used for reducing the incidence of infections of animals by pathogenic O157:H7 strains of E. coli in farm operations. The invention also relates to methods for manufacturing the biological compositions, and methods of using the biological compositions.

### 1. FIELD OF THE INVENTION

The invention relates to biological compositions comprising yeast cells that can be used to reduce the spread of pathogenic Escherichia coli infections. The invention also relates to methods for manufacturing the biological compositions, and methods of using the biological compositions.

### 2. BACKGROUND OF THE INVENTION

Escherichia coli serotype O157:H7 was only recognized as a human pathogen a little more than a decade ago, yet it has become a major foodborne pathogen. In the United States, the severity of serotype O157:H7 infections in the young and the elderly has had a tremendous impact on human health, the food industry, and federal regulations regarding food safety. In the USA, the Centers for Disease Control estimates that 73,000 cases of infection and 61 deaths in the United States can be attributed to E. coli O157:H7 annually. Most infections have been from consumption of contaminated juice, meat and other foods.

Enterohemorrhagic Escherichia coli (EHEC) serotype O157:H7 has emerged in recent years as the predominant cause of hemorrhagic colitis in humans. This illness, with characteristic symptoms of bloody diarrhea and abdominal cramps, can progress into a more severe, life-threatening complication known as hemolytic uremic syndrome (HUS). It can cause kidney failure and death, primarily in children and immune compromised adults. The pathogenicity of EHEC appears to be associated with a number of virulence factors, including the production of toxins collectively referred to as verotoxins or Shiga-like toxins. Epidemiologic investigations of past outbreaks showed that most have been associated with the consumption of bovine products. A small percentage of cattle are carriers of E. coli O257:H7. When meat is contaminated with cattle feces at slaughter, this strain of E. coli can enter the food chain. Reducing the levels of E. coli O157:H7 organisms that enter slaughter plants would require two strategies: (i) reducing the number of cattle shedding E. coli O157:H7 and (ii) reducing the magnitude of shedding by those animals infected with the organism (Cray Jr., et al., Applied Environmental Microbiology: May 1998, p.1975-1979, Vol. 64, No. 5).

Dietary management during the preslaughter period of beef production may thus play a role in reducing the incidence of E. coli O157:H7-positive ruminants. There has been conflicting information on the effect of diet on E. coli shedding from cattle. The conflict arises in part from he effect of diet on the ability of E. coli to develop acid resistance. Acid resistant bacteria are able to survive stomach acid in humans, reproduce, and produce the toxins that cause disease (Diez-Gonzalez et al., Science: Sep. 11, 1998. Volume 281, Number 5383, pages 1666-1668.) However, Hancock et al. (Science, Apr. 2, 1999 Volume 284, Number 5411, page 49) contend that this conclusion is unsupported or contradicted by several lines of evidence. The E. coli that contaminate beef typically originate from the hide, the hooves, or the equipment used in slaughter and processing rather than directly from the colon, and likely replicate in environments unlike the colon. Many in the food and agriculture industry believe that there is a need for further study of cattle feeding management practices and its use in decreasing the risk of foodborne illness from E. coli. The present invention provides a solution that uses yeasts to reduce the number of E. coli O157:H7 in the environment, which is shed by infected animals.

Citation of documents herein is not intended as an admission that any of the documents cited herein is pertinent prior art, or an admission that the cited documents are considered material to the patentability of the claims of the present application. All statements as to the date or representations as to the contents of these documents are based on the information available to the applicant and does not constitute any admission as to the correctness of the dates or contents of these documents.

### 3. SUMMARY OF THE INVENTION

The present invention relates to biological compositions that can be used to reduce the number of pathogenic Escherichia coli in or on a matter, an object or an area.

In one embodiment, the present invention provides biological compositions comprising a plurality of live yeast cells which are capable of limiting or suppressing the growth of pathogenic E. coli O157:H7 in the environment. The biological compositions can be used for reducing exposure of animals to pathogenic O157:H7 strains of E. coli.

In another embodiment, the invention provides methods of making the biological composition. In particular, the methods of the invention comprise culturing yeast cells in the presence of a series of electromagnetic fields of defined frequencies and field strengths, such that the yeast cells becomes metabolically active and potent at suppressing the growth of pathogenic E. coli. The yeast cells can also be subjected to a conditioning or acclimatizing step to improve its performance. The conditioning step comprises culturing the yeast cells in a culture medium comprising animal serum, and extracts of manure and topsoil of areas where the pathogenic bacteria are shed. Methods for manufacturing the biological compositions comprising culturing the yeast cells under activation conditions, mixing various yeast cell cultures of the present invention, followed by drying the yeast cells and packing the final product, are encompassed. In preferred embodiments, the starting yeast cells are commercially available and/or accessible to the public, such as but not limited to Saccharomyces carlsbergensis.

The biological compositions of the invention can be mixed with the environmental matter to be treated, sprinkled or spread on the object to be treated, or distributed in the area to be treated.

### 4. BRIEF DESCRIPTION OF FIGURES

FIG. 1 Activation and conditioning of yeast cells. 1 yeast cell culture; 2 container; 3 electromagnetic field source; 4 electrode.

### 5. DETAILED DESCRIPTION OF THE INVENTION

The present invention relates to biological compositions that can limit or suppress the growth of pathogenic O157:H7 strains of E. coli, reduce contamination of an object or environmental matter by pathogenic O157:H7 strains of E. coli, and reduce the incidence of infections of animals by such pathogens. The present invention provides methods for manufacturing the biological compositions as well as methods for using the biological compositions.

The biological compositions of the invention comprise live yeast cells which are distributed in environments where infections or contaminations by pathogenic E. coli are likely to occur. The use of the biological compositions of the invention can lower the overall cost of maintaining the health of animals in commercial animal operations, and reduce the contamination of meat products by pathogenic strains of E. coli.

While the following terms are believed to have well-defined meanings in the art, the following are set forth to define the terms as used herein, and facilitate explanation of the invention.

The term “animal” as used herein refers to any animal that can be infected or that can harbor pathogenic strains of E. coli. These animals are typically found in an environment where they can come in contact with the pathogenic bacteria. Examples of such animals, include farm animals and domestic pets, such as but not limited to cattle, swine, sheep, goats, horses, and poultry (chicken, duck, turkey and geese).

As used herein, the term “pathogenic E. coli” encompasses any strain of E. coli that produces a verotoxin or Shiga-like toxin. The term includes enterohemorrhagic E. coli strains and isolate O157:H7 in particular. The clonal nature of serotype O157:H7 has facilitated its phenotypic identification. Unlike other E. coli, isolates of serotype O157:H7 do not ferment sorbitol in 24 hours and are negative in the methyl-umbelliferyl glucuronide assay, which measures glucuronidase activity. These phenotypes, especially the absence of sorbitol fermentation, are used extensively to distinguish isolates of serotype O157:H7 from related bacteria. Isolation of serotype O157:H7 from foods, on selective media, such as hemorrhagic colitis agar and cefixime-tellurite sorbitol-MacConkey agar is based on the sorbitol phenotype. Since E. coli including pathogenic strains are naturally found in the gastrointestinal tracts of mammals, the contents of the gastrointestinal tracts such as animal excrements or manure, as well as the gastrointestinal tracts as animal products, are sources of the pathogenic bacteria.

The term “environment” as used herein refers to any surface or area where there is a risk of pathogenic strains of E. coli coming into contact with animals, animal products, food products for animals, food products for humans, machines and equipment for animal operations and food processing. Examples of such environments include but are not limited to farms and ranches generally, feeding areas, slaughterhouse, processing plants for animal products, storage facilities, waste product processing and disposal plants, and the like. The term encompasses the facilities and equipment in all phases of industrialized animal operations where many different functions are centralized and where cross-contamination is highly likely.

In one embodiment, the present invention provides biological compositions that comprise a population of live yeast cells which have been cultured under a specific set of conditions such that the yeast cells are capable of suppressing the growth of pathogenic E. coli.

According to the invention, under certain specific culture conditions, yeasts can be made metabolically active such that they become effective and potent at suppressing the growth of pathogenic bacteria in their vicinity. Without being bound by any theory or mechanism, the inventor believes that the culture conditions activate and/or amplified the expression of a gene or a set of genes in the yeast cells such that the metabolism of the yeast cells becomes highly active. It is envisioned that, due to the high metabolic activity of the yeasts after they have been cultured under the conditions described hereinbelow, interactions between certain yeast gene products and the pathogenic bacteria cause the pathogens to lose viability. It is also envisioned that the pathogenic bacteria cannot compete with the yeast cells for essential nutrients and hence, fail to grow normally or grow at a lower rate. As a result of these interactions, the ability of E. coli O157:H7 cells to proliferate is suppressed and the spread of the bacteria in the environment is contained. The animals or parts of the slaughtered animals are thus less likely to be exposed to and become infected with pathogenic E. coli.

In one embodiment, the biological compositions of the invention can be added to or mixed with a potential or an actual source of pathogenic E. coli. In another embodiment, an object which may become or which may have been contaminated with pathogenic E. coli is contacted with the biological compositions of the invention. In yet another embodiment of the invention, the biological compositions can be distributed over an area or space which may become contaminated with or is a source of pathogenic E. coli. As known to those skilled in the relevant art, many methods and appliances may be used to mix the biological compositions of the invention with a potential source of the pathogen, such as animal waste, manure, sludge, and sewage. In a particular embodiment, a mixture of culture broths of the yeasts of the present invention are added directly to such sources. Dried powders of the yeasts can also be used. The yeast cells are distributed, or sprinkled or spreaded onto environmental matters (e.g., contaminated soil), an object, or an area to the treated. The biological compositions may be applied to and/or distributed by any mechanized means which may be automated.

The amount of biological composition used depends in part on the mode of use which can be determined empirically. Although not necessary, the biological compositions of the invention can also be used in conjunction or in rotation with other types of decontaminating agents, provided that they do not kill the yeast cells or render it impossible to sustain yeast cell growth.

Described below in Section 5.1 and 5.2 are four yeast cell components of the invention and methods of their preparation. Section 5.3 describes the manufacture of the biological compositions of the invention which comprises at least one of the four yeast cell components.

### 5.1 PREPARATION OF THE YEAST CELL CULTURES

In one embodiment, the present invention provides yeast cells that are capable of suppressing the growth of pathogenic E. coli O157:H7.

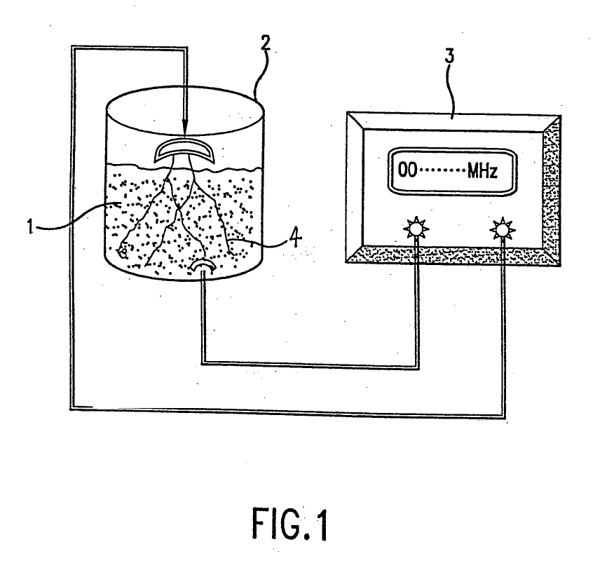

The yeast cells of the invention are prepared by culturing in an appropriate culture medium in the presence of an alternating electromagnetic field or multiple alternating electromagnetic fields in series over a period of time. The culturing process allows yeast spores to germinate, yeast cells to grow and divide, and can be performed as a batch process or a continuous process. As used herein, the terms “alternating electromagnetic field”, “electromagnetic field” or “EM field” are synonymous. An electromagnetic field useful in the invention can be generated by various means well known in the art. A schematic illustration of exemplary setups are depicted respectively in FIG. 1. An electromagnetic field of a desired frequency and a desired field strength is generated by an electromagnetic wave source (3) which comprises one or more signal generators that are capable of generating electromagnetic waves, preferably sinusoidal waves, and preferably in the frequency range of 30 MHz-20,000 MHz. Such signal generators are well known in the art. Signal generators capable of generating signal with a narrower frequency range can also be used. If desirable, a signal amplifier can also be used to increase the output signal, and thus the strength of the EM field.

The electromagnetic field can be applied to the culture by a variety of means including placing the yeast cells in close proximity to a signal emitter connected to a source of electromagnetic waves. In one embodiment, the electromagnetic field is applied by signal emitters in the form of electrodes that are submerged in a culture of yeast cells (1). In a preferred embodiment, one of the electrodes is a metal plate which is placed on the bottom of a non-conducting container (2), and the other electrode comprises a plurality of wires or tubes so configured inside the container such that the energy of the electromagnetic field can be evenly distributed in the culture. The tips of the wires or tubes are placed within 3 to 30 cm from the bottom electrode plate (i.e, approximately 2 to 10% of the height of the container from the bottom). The number of electrode wires used depends on both the volume of the culture and the diameter of the wire. For example, for a culture having a volume of 10 liter or less, two ro three electrode wires having a diameter of between 0.5 to 2.0 mm can be used. For each 100 liter to 1000 liter of culture, the electrode wires or tubes can have a diameter of 6.0 to 15.0 mm. For a culture having a volume greater than 1000 liter, the electrode wires or tubes can have a diameter of between 20.0 to 25.0 mm.

In various embodiments, yeasts of the genera of Saccharomyces, Candida, Crebrothecium, Geotrichum, Hansenula, Kloeckera, Lipomyces, Pichia, Rhodosporidium, Rhodotorula Torulopsis, Trichosporon, and Wickerhamia can be used in the invention.

Non-limiting examples of yeast strains include Saccharomyces cerevisiae Hansen, ACCC2034, ACCC2035, ACCC2036, ACCC2037, ACCC2038, ACCC2039, ACCC2040, ACCC2041, ACCC2042, AS2.1, AS2.4, AS2.1 1, AS2.14, AS2.16, AS2.56, AS2.69, AS2.70, AS2.93, AS2.98, AS2.101, AS2.109, AS2.110, AS2.112, AS2.139, AS2.173, AS2.174, AS2.182, AS2.196, AS2.242, AS2.336, AS2.346, AS2.369, AS2.374, AS2.375, AS2.379, AS2.380, AS2.382, AS2.390, AS2.393, AS2.395, AS2.396, AS2.397, AS2.398, AS2.399, AS2.400, AS2.406, AS2.408, AS2.409, AS2.413, AS2.414, AS2.415, AS2.416, AS2.422, AS2.423, AS2.430, AS2.431, AS2.432, AS2.451, AS2.452, AS2.453, AS2.458, AS2.460, AS2.463, AS2.467, AS2.486, AS2.501, AS2.502, AS2.503, AS2.504, AS2.516, AS2.535, AS2.536, AS2.558, AS2.560, AS2.561, AS2.562, AS2.576, AS2.593, AS2.594, AS2.614, AS2.620, AS2.628, AS2.631, AS2.666, AS2.982, AS2.1190, AS2.1364, AS2.1396, IFFI 1001, IFFI 1002, IFFI 1005, IFFI 1006, IFFI 1008, IFFI 1009, IFFI 1010, IFFI 1012, IFFI 1021, IFFI 1027, IFFI 1037, IFFI 1042, IFFI 1043, IFFI 1045, IFFI 1048, IFFI 1049, IFFI 1050, IFFI 1052, IFFI 1059, IFFI 1060, IFFI 1063, IFFI 1202, IFFI 1203, IFFI 1206, IFFI 1209, IFFI 1210, IFFI 1211, IFFI 1212, IFFI 1213, IFFI 1215, IFFI 1220, IFFI 1221, FFI 1224, IFFI 1247, IFFI 1248, FFI 1251, IFFI 1270, IFFI 1277, IFFI 1287, IFFI 1289, IFFI 1290, IFFI 1291, IFFI 1292, IFFI 1293, IFFI 1297, IFFI 1300, IFFI 1301, IFFI 1302, IFFI 1307, IFFI 1308, IFFI 1309, IFFI 1310, IFFI 1311, FFI 1331, FFI 1335, FFI 1336, IFFI 1337, IFFI 1338, IFFI 1339, IFFI 1340, IFFI 1345, IFFI 1348, IFFI 1396, IFFI 1397, IFFI 1399, IFFI 1411, IFFI 1413; Saccharomyces cerevisiae Hansen Var. ellipsoideus (Hansen) Dekker, ACCC2043, AS2.2, AS2.3, AS2.8, AS2.53, AS2.163, AS2.168, AS2.483, AS2.541, AS2.559, AS2.606, AS2.607, AS2.61 1, AS2.612; Saccharomyces chevalieri Guillermond, AS2.131, AS2.213; Saccharomyces delbrueckii, AS2.285; Saccharomyces delbrueckii Lindner var. mongolicus Lodder et van Rij, AS2.209, AS2.1157; Saccharomyces exiguous Hansen, AS2.349, AS2.1158; Saccharomyces fermentati (Saito) Lodder et van Rij, AS2.286, AS2.343; Saccharomyces logos van laer et Denamur ex Jorgensen, AS2.156, AS2.327, AS2.335; Saccharomyces mellis Lodder et Kreger Van Rij, AS2.195; Saccharomyces microellipsoides Osterwalder, AS2.699; Saccharomyces oviformis Osterwalder, AS2.100; Saccharomyces rosei (Guilliermond) Lodder et kreger van Rij, AS2.287; Saccharomyces rouxii Boutroux, AS2.178, AS2.180, AS2.370, AS2.371; Saccharomyces sake Yabe, ACCC2045; Candida arborea, AS2.566; Candida Krusei (Castellani) Berkhout, AS2.1045; Candida lambica(Lindner et Genoud) van. Uden et Buckley, AS2.1182; Candida lipolytica (Harrison) Diddens et Lodder, AS2.1207, AS2.1216, AS2.1220, AS2.1379, AS2.1398, AS2.1399, AS2.1400; Candida parapsilosis (Ashford) Langeron et Talice, AS2.590; Candida parapsilosis (Ashford) et Talice Var. intermedia Van Rij et Verona, AS2.491; Candida pulcherriman (Lindner) Windisch, AS2.492; Candida rugousa (Anderson) Diddens et Loddeer, AS2.511, AS2.1367, AS2.1369, AS2.1372, AS2.1373, AS2.1377, AS2.1378, AS2.1384; Candida tropicalis (Castellani) Berkout, ACCC2004, ACCC2005, ACCC2006, AS2.164, AS2.402, AS2.564, AS2.565, AS2.567, AS2.568, AS2.617, AS2.1387; Candida utilis Henneberg Lodder et Kreger Van Rij, AS2.120, AS2.281, AS2.1180; Crebrothecium ashbyii (Guillermond) Routein, AS2.481, AS2.482, AS2.1197; Geotrichum candidum Link, ACCC2016, AS2.361, AS2.498, AS2.616, AS2.1035, AS2.1062, AS2.1080, AS2.1132, AS2.1175, AS2.1183; Hansenula anomala (Hansen) H et P sydow, ACCC2018, AS2.294, AS2.295, AS2.296, AS2.297, AS2.298, AS2.299, AS2.300, AS2.302, AS2.338, AS2.339, AS2.340, AS2.341, AS2.470, AS2.592, AS2.641, AS2.642, AS2.635, AS2.782, AS2.794; Hansenula arabitolgens Fang, AS2.887; Hansenula jadinii Wickerham, ACCC2019; Hansenula saturnus (Klocker) H et P sydow, ACCC2020; Hansenula schneggii (Weber) Dekker, AS2.304; Hansenula subpelliculosa Bedford, AS2.738, AS2.740, AS2.760, AS2.761, AS2.770, AS2.783, AS2.790, AS2.798, AS2.866; Kloeckera apiculata (Reess emend. Klocker) Janke, ACCC2021, ACCC2022, ACCC2023, AS2.197, AS2.496, AS2.711, AS2.714; Lipomyces starkeyi Lodder et van Rij, ACCC2024, AS2.1390; Pichia farinosa (Lindner) Hansen, ACCC2025, ACCC2026, AS2.86, AS2.87, AS2.705, AS2.803; Pichia membranaefaciens Hansen, ACCC2027, AS2.89, AS2.661, AS2.1039; Rhodosporidium toruloides Banno, ACCC2028; Rhodotorula glutinis (Fresenius) Harrison, ACCC2029, AS2.280, ACCC2030, AS2.102, AS2.107, AS2.278, AS2.499, AS2.694, AS2.703, AS2.704, AS2.1146; Rhodotorula minuta (Saito) Harrison, AS2.277; Rhodotorula rubar (Demme) Lodder, ACCC2031, AS2.21, AS2.22, AS2.103, AS2.105, AS2.108, AS2.140, AS2.166, AS2.167, AS2.272, AS2.279, AS2.282; Saccharomyces carlsbergensis Hansen, AS2.113, ACCC2032, ACCC2033, AS2.312, AS2.116, AS2.118, AS2.121, AS2.132, AS2.162, AS2.189, AS2.200, AS2.216, AS2.265, AS2.377, AS2.417, AS2.420, AS2.440, AS2.441, AS2.443, AS2.444, AS2.459, AS2.595, AS2.605, AS2.638, AS2.742, AS2.745, AS2.748, AS2.1042; Saccharomyces uvarum Beijer, IFFI 1023, IFFI 1032, IFFI 1036, IFFI 1044, IFFI 1072, IFFI 1205, IFFI 1207; Saccharomyces willianus Saccardo, AS2.5, AS2.7, AS2.119, AS2.152, AS2.293, AS2.381, AS2.392, AS2.434, AS2.614, AS2.1189; Saccharomyces sp., AS2.311; Saccharomyces ludwigii Hansen, ACCC2044, AS2.243, AS2.508; Saccharomyces sinenses Yue, AS2.1395; Schizosaccharomyces octosporus Beijerinck, ACCC 2046, AS2.1148; Schizosaccharomyces pombe Linder, ACCC2047, ACCC2048, AS2.248, AS2.249, AS2.255, AS2.257, AS2.259, AS2.260, AS2.274, AS2.994, AS2.1043, AS2.1149, AS2.1178, IFFI 1056; Sporobolomyces roseus Kluyver et van Niel, ACCC 2049, ACCC 2050, AS2.619, AS2.962, AS2.1036, ACCC2051, AS2.261, AS2.262; Torulopsis candida (Saito) Lodder, ACCC2052, AS2.270; Torulopsis famta (Harrison) Lodder et van Rij, ACCC2053, AS2.685; Torulopsis globosa (Olson et Hammer) Lodder et van Rij, ACCC2054, AS2.202; Torulopsis inconspicua Lodder et van Rij, AS2.75; Trichosporon behrendii Lodder et Kreger van Rij, ACCC2055, AS2.1193; Trichosporon capitatum Diddens et Lodder, ACCC2056, AS2.1385; Trichosporon cutaneum(de Beurm et al.)Ota, ACCC2057, AS2.25, AS2.570, AS2.571, AS2.1374; Wickerhamia fluoresens (Soneda) Soneda, ACCC2058, AS2.1388. Yeasts of the Saccharomyces genus are generally preferred. Saccharomyces cerevisiae and Saccharomyces carlsbergensis are preferred strains.

Generally, yeast strains useful for the invention can be obtained from private or public laboratory cultures, or publically accessible culture deposits, such as the American Type Culture Collection, 10801 University Boulevard, Manassas, Va. 20110-2209 and the China General Microbiological Culture Collection Center (CGMCC), China Committee for Culture Collection of Microorganisms, Institute of Microbiology, Chinese Academy of Sciences, Haidian, P.O. Box 2714, Beijing, 100080, China.

Although it is preferred, the preparation of the yeast cell components of the invention is not limited to starting with a pure strain of yeast. Each yeast cell component may be produced by culturing a mixture of yeast cells of different species or strains. The constituents of a yeast cell component can be determined by standard yeast identification techniques well known in the art.

In various embodiments of the invention, standard techniques for handling, transferring, and storing yeasts are used. Although it is not necessary, sterile conditions or clean environments are desirable when carrying out the manufacturing processes of the invention. Standard techniques for handling animal blood and immune cells, and for studying immune functions of an animal are also used. Details of such techniques are described in Advances in Laboratory Methods: General Haematology, 2000, Assendelft et al., (Ed.), Arnold, Edward (Publisher); Handbook of Vertebrate Immunology, 1998, Pastoret et al. (Ed.), Academic Press, and Current Protocols In Immunology, 1991, Coligan, et al. (Ed), John Wiley & Sons, Inc., which are both incorporated herein by reference in their entireties.

In one embodiment, the yeast cells are first cultured in the presence of at least one alternating electromagnetic (EM) field with a frequency in the range of 10540 MHz to 10560 MHz. A single EM field or a first series of EM fields can be applied, each having a different frequency within the stated range, or a different field strength within the stated range, or different frequency and field strength within the stated ranges. Although any practical number of EM fields can be used within a series, it is preferred that, the yeast culture be exposed to a total of 2, 3, 4, 5, 6, 7, 8, 9 or 10 different EM fields in a series. The EM field(s), which can be applied by any means known in the art, can each have a frequency of 10540, 10541, 10542, 10543, 10544, 10545, 10546, 10547, 10548, 10549, 10550, 10551, 10552, 10553, 10554, 10555, 10556, 10557, 10558, 10559, or 10560 MHz. The field strength of the EM field(s) is in the range of 65 to 255 mV/cm preferably 212±2.0 MV/cm. The yeast cells can be cultured in the EM fields for 36 to 136 hours. The yeast culture can remain in the same container and use the same set of electromagnetic wave generator and emitters when switching from one EM field to another EM field.

The culture process can be initiated by inoculating 1000 ml of medium with an inoculum of the selected yeast strain(s) of about 10⁸ cells. The starting culture is kept at 28±1° C. for 24 to 56 hours prior to exposure to the EM field(s). The culturing process may preferably be conducted under conditions in which the concentration of dissolved oxygen is between 0.025 to 0.08 mol/m³, preferably 0.04 mol/m³. The oxygen level can be controlled by any conventional means known in the art, including but not limited to stirring and/or bubbling.

The culture is most preferably carried out in a liquid medium which contains animal serum and sources of nutrients assimilable by the yeast cells. Table 1 provides an exemplary medium for culturing the first yeast cell component of the invention.

TABLE 1

| Medium Composition | Quantity | |
|---|---|---|
| Mannitol | 18.0 | g |
| K₂HPO₄ | 0.2 | g |
| MgSO₄.7H₂O | 0.3 | g |
| NaCl | 0.3 | g |
| CaSO₄.2H₂O | 0.2 | g |
| CaCO₃.5H₂O | 4.0 | g |
| Peptone | 1.2 | g |
| Bovine serum | 400 | ml |
| Autoclaved water | 600 | ml |

In general, carbohydrates such as sugars, for example, sucrose, glucose, fructose, dextrose, maltose, xylose, and the like and starches, can be used either alone or in combination as sources of assimilable carbon in the culture medium. The exact quantity of the carbohydrate source or sources utilized in the medium depends in part upon the other ingredients of the medium but, in general, the amount of carbohydrate usually varies between about 0.1% and 5% by weight of the medium and preferably between about 0.5% and 2%, and most preferably about 0.8%. These carbon sources can be used individually, or several such carbon sources may be combined in the medium. Among the inorganic salts which can be incorporated in the culture media are the customary salts capable of yielding sodium, calcium, phosphate, sulfate, carbonate, and like ions. Non-limiting examples of nutrient inorganic salts are (NH₄)₂HPO₄, CaCO₃, MgSO₄, NaCl, and CaSO₄.

The bovine serum is a fraction of blood that comprises white blood cell, and can be prepared from whole blood (1000-2000 ml) by standard methods known in the art, such as density gradient centrifugation. Red blood cells are separated and discarded. The serum may be diluted or concentrated. The serum is added to the culture medium after the medium has been autoclaved and cooled to about 45° C.

It should be noted that the composition of the media provided in Table 1 is not intended to be limiting. The process can be scaled up or down according to needs. Various modifications of the culture medium may be made by those skilled in the art, in view of practical and economic considerations, such as the scale of culture and local supply of media components.

Although the yeast cells will become activated even after a few hours of culturing in the presence of the EM field(s), the yeast cells can be cultured in the presence of the EM field(s) for an extended period of time (e.g., one or more weeks). At the end of the culturing process, the yeast cells which constitute the first yeast cell component of the invention may be recovered from the culture by various methods known in the art, and stored at a temperature below about 0° C. to 4° C. The recovered yeast cells may also be dried and stored in powder form.

A non-limiting example of making the yeast cells of the invention with Saccharomyces cerevisiae strain AS2.504 is provided in Section 6 hereinbelow. Saccharomyces carlsbergensis strain AS2.605 is also preferred as a starting cell strain for making the yeast cells of the invention.

After having been cultured in a first series of EM fields, the yeast cells are subjected to culturing in the presence of at least a second alternating electromagnetic (EM) field with a frequency in the range of 13210 MHz to 13230 MHz. A single EM field or a second series of EM fields can be applied, each having a different frequency within the stated range, or a different field strength within the stated range, or different frequency and field strength within the stated ranges. Although any practical number of EM fields can be used within a series, it is preferred that, the yeast culture be exposed to a total of 2, 3, 4, 5, 6, 7, 8, 9 or 10 different EM fields in a series. The EM field(s), which can be applied by any means known in the art, can each have a frequency of 13210, 13211, 13212, 13213, 13214, 13215, 13216, 13217, 13218, 13219, 13220, 13221, 13222, 13223, 13224, 13225, 13226, 13227, 13228, 13229, or 13230 MHz. The field strength of the EM field(s) is in the range of 80 to 190 mV/cm preferably at 169±4.0 mV/cm. The yeast cells can be cultured in the EM fields for 36 to 96 hours. The yeast culture can remain in the same container and use the same set of electromagnetic wave generator and emitters when switching from one EM field to another EM field.

The culture process can be initiated by inoculating 1000 ml of medium with an inoculum of the selected yeast strain(s) of about 10⁸ cells. The starting culture is kept at 28±1° C. for 24 to 56 hours prior to exposure to the EM field(s). The culturing process may preferably be conducted under conditions in which the concentration of dissolved oxygen is between 0.025 to 0.08 mol/m³, preferably 0.04 mol/m³. The oxygen level can be controlled by any conventional means known in the art, including but not limited to stirring and/or bubbling.

The culture is most preferably carried out in a liquid medium which contains animal serum and sources of nutrients assimilable by the yeast cells. Table 2 provides an exemplary medium for culturing the second yeast cell component of the invention.

TABLE 2

| Medium Composition | Quantity | |
|---|---|---|
| Sucrose | 20 | g |
| K₂HPO₄ | 0.2 | g |
| MgSO₄.7H₂O | 0.3 | g |
| NaCl | 0.2 | g |
| CaSO₄.2H₂O | 0.3 | g |
| CaCO₃.5H₂O | 3.0 | g |
| Yeast extract | 0.8 | g |
| Bovine serum | 500 | ml |
| Autoclaved water | 500 | ml |

In general, carbohydrates such as sugars, for example, sucrose, glucose, fructose, dextrose, maltose, xylose, and the like and starches, can be used either alone or in combination as sources of assimilable carbon in the culture medium. The exact quantity of the carbohydrate source or sources utilized in the medium depends in part upon the other ingredients of the medium but, in general, the amount of carbohydrate usually varies between about 0.1% and 5% by weight of the medium and preferably between about 0.5% and 2%, and most preferably about 0.8%. These carbon sources can be used individually, or several such carbon sources may be combined in the medium. Among the inorganic salts which can be incorporated in the culture media are the customary salts capable of yielding sodium, calcium, phosphate, sulfate, carbonate, and like ions. Non-limiting examples of nutrient inorganic salts are (NH₄)₂HPO₄, CaCO₃, MgSO₄, NaCl, and CaSO₄.

It should be noted that the composition of the media provided in Table 2 is not intended to be limiting. The process can be scaled up or down according to needs. Various modifications of the culture medium may be made by those skilled in the art, in view of practical and economic considerations, such as the scale of culture and local supply of media components.

Although the yeast cells will become activated even after a few hours of culturing in the presence of the EM field(s), the yeast cells can be cultured in the presence of the EM field(s) for an extended period of time (e.g., one or more weeks). At the end of the culturing process, the yeast cells which constitute the second yeast cell component of the invention may be recovered from the culture by various methods known in the art, and stored at a temperature below about 0° C. to 4° C. The recovered yeast cells may also be dried and stored in powder form.

### 5.2 CONDITIONING OF THE YEAST CELLS

According to the invention, performance of the activated yeast cells can be optimized by culturing the activated yeast cells in the presence of a mixture comprising animal serum, an extract of animal manure and an extract of topsoil of an area which is or can be contaminated with pathogenic E. coli O157:H7. The inclusion of this additional conditioning or acclimatizing process allows the activated yeast cells to adapt to and endure the physical environment in which the yeast cells are expected to remain metabolically active. The method for conditioning or acclimatizing activated yeast cells of the invention comprises culturing yeast cells in the mixture as described below in an alternating electromagnetic (EM) field.

The culture process can be initiated by inoculating 1000 ml of a conditioning medium with about 10 ml of activated yeasts containing about 10⁸ cells/ml (as prepared by the methods described in section 5.1). The initial number of yeast cells in the conditioning medium is about 10⁶ cells/ml. A equivalent number of dried yeast cells can also be used as an inoculum. The conditioning medium comprises per 1000 ml about 200 ml of animal serum, such as bovine serum, about 500 ml of manure extract, and about 300 ml of topsoil extract. The process can be scaled up or down according to needs.

The animal serum can be prepared as described in Section 5.1. Typically, bovine serum is used.

The manure extract is prepared by mixing about 1000 g of animal manure, such as cattle manure, with 3000 ml of water, incubating the mixture for 24 hours at room temperature, and filtering the mixture to remove particulate matters. The clarified liquid is collected and kept at 4° C. Other methods that can be used to collect the extract from the mixture include centrifugation of the mixture. Preferably, the collection procedures and storage are carried out under clean or sterile conditions.

The topsoil extract is prepared by mixing about 1000 g of topsoil, such as soil from the surface of an area in a ranch or farm where animals shed their waste products, with 3000 ml of water, incubating the mixture for 24 hours at room temperature, and filtering the mixture to remove particulate matters. The clarified liquid is collected and kept at 4° C. Other methods that can be used to collect the extract from the mixture include centrifugation of the mixture. Preferably, the collection procedures and storage are carried out under clean or sterile conditions.

The activated yeast cells are cultured in conditioning medium in the presence of an EM field. The frequency of the EM field is 17557 MHz. The field strength is in the range of 80 to 230 mV/cm, preferably at 203±2 mV/cm. About 10⁹ activated yeast cells of the first yeast cell component are added to 1000 ml of conditioning medium. The temperature is maintained at 28±2° C. The yeast culture is exposed to the EM field for about 58 hours.

The activated and conditioned yeast cells may be recovered from the culture by various methods known in the art, and preferably stored in powder form at a temperature below about 0° C. to 4° C. The powder form of the yeast cells comprises greater than about 10⁹ to 10¹⁰ yeast cells per gram. The activated and conditioned yeast cells can be dried as follows: the yeast cells was dried in a first dryer at a temperature not exceeding 60±2° C. for a period of 5 minutes so that yeast cells quickly became dormant. The yeast cells were then sent to a second dryer and dried at a temperature not exceeding 65±2° C. for a period of about 8 minutes to further remove water. The dried yeast cells were then cool to room temperature.

The activated and conditioned yeast cells can be used immediately, stored for later use, or used as a starter culture for large scale manufacturing.

### 5.3 MANUFACTURE OF THE BIOLOGICAL COMPOSITIONS

The present invention also encompasses methods of manufacturing of the biological compositions of the invention. The activated and conditioned yeast cells as prepared by section 5.1 and 5.2 can be propagated on a large scale to make the biological compositions of the invention. The method comprises culturing the yeast cells in the presence of two series of EM fields for a period of time, diluting the growing yeast cells with fresh medium, and repeating the process. The method can be carried out as a batch process or a continuous process.

In one preferred embodiment, a set of three containers (5, 6, 7) each comprising a set of electrodes for generating an electromagnetic field as described in section 5.1 and 5.2 are set up each with 1000 liters of a culture medium. See FIG. 2. The culture medium comprises nutrients assimilable by the yeast cells as shown in Table 3.

TABLE 3

| Material | Quantity | |
|---|---|---|
| Starch | 20 | kg |
| peptone | 3 | kg |
| Distilled water | 1000 | liters |

The first container is inoculated with activated and conditioned yeast cells at about 1×10⁸ cell/ml. For example, a 1000 liter container is used which has a height to diameter ratio of 1.3 to 1. The yeast cells are then subjected to a EM field. The frequency used is 17557 MHz (up to ±2.3 MHz) at 187 mV/cm (useable range is 80 to 230 mV/cm). The temperature is maintained at 26 to 30° C., preferably at 28° C. The yeast culture is exposed to the EM field for about 56-72 hours, preferably 68° C. The yeast cells in the first container is used as a seed culture to inoculate the culture medium in the second container. About 5 ml of yeast cells in the first container is added per 100 ml of culture medium in the second container. The yeast cells in the second container are then subjected to an EM field. The frequency is also 17557 MHz. The field strength is in the range of 80 to 230 mV/cm. The temperature is maintained at 28±2° C. The yeast culture is exposed to the EM field for about 56-72 hours. After culturing in the second container, the yeast cells are transferred into a third container typically when a density of about 10⁹ cells/ml is reached.

The yeast cell culture resulting from the end of this stage can be used directly as a biological composition of the invention. Preferably, a biological composition comprising the yeast cells is prepared by adsorbing the yeast cells to an absorbent carrier. To facilitate this, the yeast cell culture is concentrated using methods known in the art, such as drying under vaccum. The concentration process is carried out in two stages. At the first stage, the volume of the liquid culture is reduced to about 80% of the original volume. During the second stage, the volume is reduced from 80% to 72% and finally to about 50%. The biological composition can be prepared by mixing the yeast cells with an absorbent carrier such as starch or zeolite powder (less than 200 mesh) at a ratio of 100 to 120 ml of concentrated yeast cells (5-10 kg dried cells) per 980 to 990 kg of carrier to make 1000 kg of the composition. The mixture is dried at a temperature not exceeding 70° C. for a period of time less than 10 minutes such that the yeast cells become dormant, and the moisture content is below 5%. The final dried product comprises greater than or equal to about 10⁸ yeasts per gram.

To use the biological composition, the dried yeast cells are mixed with water in a range of ratios and applied to the composition, object, or environment which is or may become contaminated with pathogenic E. coli, for example, 1:30, biological composition to water by weight.

### 6. EXAMPLE

The following example illustrates the making and testing of a biological composition that can be used to control the spread of E. coli O157:H7. The biological composition comprises Saccharomyces cerevisiae strain AS2.504 cells which have been activated by the procedure described in section 5.1. The yeast cells were prepared in two stages and tested as follows:

A starting culture containing about 10⁵ cells/ml of AS2.504 was placed into the container (2) as shown in FIG. 1. The medium had the composition shown in Table 1. Initially, the yeast cells were cultured for about 52 hours at 28±1° C. without an EM field. Then, in the same medium, at 28±1° C., the yeast cells were cultured in the presence of a first series of four EM fields applied in the order stated: 10544 MHz at 212 mv/cm for 10 hrs; 10550 MHz at 212 mv/cm for 10 hrs; 10553 MHz at 212 mv/cm for 42 hrs; and 10559 MHz at 212mv/cm for 58 hrs.

The AS2.504 yeast cells were then subjected to a second series of EM fields. A culture containing about 10⁶ cells/ml of activated AS2.504 cells was placed into the container (2) as shown in FIG. 1. 10 ml of activated yeast cells containing 10⁸ cells/ml is added to a 1000 ml of medium. The medium has a composition as shown in Table 2. Initially, the yeast cells are cultured for about 24 to 56 hours at 28° C. without an EM field. Then, in the same medium, at 28° C., the yeast cells were cultured in the presence of a second series of four EM fields applied in the order stated: 13210 MHz at 169 mv/cm for 36 hrs; 13214 MHz at 169 mv/cm for 36 hrs; 13219 MHz at 169 mv/cm for 12 hrs; 13226 MHz at 169 mv/cm for 12 hrs.

The activated yeast cells were tested for their effect on E. coli O157:H7 cells in vitro. Three aliquots of bovine serum, 200 ml each, was obtained from a cattle (of a Dutch breed kept for meat) under sterile conditions. 0.1 ml of activated AS 2.504 yeast cells (10⁹ per ml) were added to a first aliquot of bovine serum (Group A) and the mixture was kept in a tissue culture incubator at 28±2° C. under CO₂ for about 24 hours. 0.1 ml of non-activated AS 2.504 yeast cells (10⁹ per ml) were added to another aliquot of bovine serum (Group B) and the mixture were kept under the same conditions as Group A. No yeast cells were added to the last aliquot of bovine serum (Group C) which were kept under the same conditions as the other groups and acted as a negative control.

After 24 hours of incubation, the concentration of E. coli O157:H7 cells in each Group were determined. The results are shown in Table 4 below.

TABLE 4

| | Concentration of | Percentage Decrease |
|---|---|---|
| | live E. coli | in concentration |
| Experimental Groups | after incubation | of E. coli |
| Activated AS2.504: Group A | 0.01 × 10⁸/ml | 99.99% |
| Non-activated AS2.504: | 12.19 × 10⁸/ml | 0.65% |
| Group B | | |
| No yeast cells: Group C | 12.27 × 10⁸/ml | 0% |

The results indicate that activated AS2.504 cells can reduce the concentration of E. coli cells as compared to non-activated AS2.504 cells. In the presence of the yeast cells of the invention, the growth of E. coli cells is reduced or suppressed.

The cells activated as described above were further cultured in a conditioning medium as described in Table 2 and in the presence of an EM field. The frequency used was 17557 MHz and the field strength was 203 mV/cm. The culturing period was about 58 hours while the temperature was maintained at 28° C. After the culture period is over, the yeast culture were concentrated and dried as described in Section 5.3 to form the biological composition of the invention.

The activated and conditioned AS2.504 yeast cells were made into a biological composition and tested for their effect on E. coli O157:H7 cells in vitro. 20 ml of a stock of E. coli O157:H7 cells (at 10⁹/ml) was mixed with 600 ml of sterile water. The mixture was divided into two aliquots each of which were sprayed separately onto 1000 g of cattle manure in a ceramic container. Both containers were incubated at 28° C. for 24 hours. Before adding the biological composition, 2 g of the composition is mixed with 60 ml of sterile water. The composition was then sprayed onto one container (A). The other container (B) did not receive any biological composition. Both containers were incubated at 28±1° C. for 48 hours and the numbers of live E. coli O157:H7 cells from each container were determined by standard methods. In container B, the number of live E. coli O157:H7 cells were 12.53×10⁶ cells/g of cattle manure. No live E. coli O157:H7 cells from container A were detected in the serial dilutions used for counting the cells from container B. The results indicated that the number of E. coli O157:H7 cells (which had been co-cultiavted with yeast cells of the invention) was significantly less in container A than in container B, and that the biological composition is capable of suppressing the growth of pathogenic E. coli cells.

The present invention is not to be limited in scope by the specific embodiments described which are intended as single illustrations of individual aspects of the invention, and functionally equivalent methods and components are within the scope of the invention. Indeed various modifications of the invention, in addition to those shown and described herein will become apparent to those skilled in the art from the foregoing description and accompanying drawings. Such modifications are intended to fall within the scope of the appended claims.

What is claimed is:

1. A biological composition comprising a plurality of yeast cells that are prepared by culturing the yeast cells in
(a) a first electromagnetic field or a first series of electromagnetic fields having a frequency in the range of 10540 to 10560 MHz and a field strength of 65 to 255 mV/cm; and
(b) a second electromagnetic field or a second series of electromagnetic fields having a frequency in the range of 13210 to 13230 MHz and a field strength of 80 to 190 mV/cm.

2. The biological composition of claim 1 wherein the yeast cells are further cultured in a culture medium comprising animal serum, a manure extract and a topsoil extract in the presence of a third electromagnetic field or a third series of electromagnetic fields having a frequency of 17557 MHz and a field strength of 80 to 230 mV/cm.

3. The biological composition of claim 1 or 2, wherein the yeast cells are cells of Saccharomyces.

4. The biological composition of claim 1 or 2, wherein the yeast cells are cells of Saccharomyces cerevisiae or Saccharomyces carlsbergensis.

5. The biological composition of claim 1 or 2 in which the yeast cells are dried.

6. A composition comprising the biological composition of claim 1 or 2, and a carrier.

7. The composition of claim 6 wherein the carrier is zeolite powder at a ratio of about 10⁸ yeast cells to 1 g of zeolite powder.

8. The composition of claim 1 or 2, wherein the plurality of yeast cells are cells of Saccharomyces carlsbergensis AS2.605, and/or cells of Saccharomyces cerevisiae AS2.504.

9. A method for preparing a biological composition, said method comprising culturing a plurality of yeast cells in an electromagnetic field or a series of electromagnetic fields having a frequency in the range of 10540 to 10560 MHz and a field strength of 65 to 255 mV/cm.

10. The method of claim 9, wherein said method further comprises culturing the plurality of yeast cells in one or more of the electromagnetic fields in a culture medium comprising animal serum, a manure extract and a topsoil extract.

11. A method for preparing a biological composition, said method comprising culturing a plurality of yeast cells in an electromagnetic field or a series of electromagnetic fields having a frequency in the range of 13210 to 13230 MHz and a field strength of 80 to 190 mV/cm.

12. The method of claim 11, wherein said method further comprises culturing the plurality of yeast cells in one or more of the electromagnetic fields in a culture medium comprising animal serum, a manure extract and a topsoil extract.

13. A method of making a biological composition, said method comprising
(a) preparing the yeast cells of claim 1, and
(b) adsorbing the yeast cells of (a) to a carrier.

14. The method of claim 13, wherein the adsorbing step comprises (i) concentrating the yeast cell culture by about 50%, (ii) mixing the yeast cell culture with the carrier; and (iii) drying the mixture at a temperature not exceeding 70° C. to reduce the moisture content to below 5%.

15. A method for suppressing the growth of Escherichia coli O157:H7 in a composition or in an area comprising contacting the composition or the area for a period of time with a biological composition comprising yeast cells having been cultured in:
(a) a first electromagnetic field or a first series of electromagnetic fields having a frequency in the range of 10540 to 10560 MHz and a field strength of 65 to 255 mV/cm;
(b) a second electromagnetic field or a second series of electromagnetic fields having a frequency in the range of 13210 to 13230 MHz and a field strength of 80 to 190 mV/cm; and
(c) a third electromagnetic field or a third series of electromagnetic fields having a frequency of 17557 MHz and a field strength of 80 to 203 mV/cm.

16. The method of claim 15, wherein the biological composition comprises yeast cells and zeolite powder.

17. The method of claim 15, wherein said yeast cells are Saccharomyces carlsbergensis or Saccharomyces cerevisiae cells.

18. The method of claim 15, wherein the plurality of yeast cells are cells of Saccharomyces carlsbergensis AS2.605, and/or cells of Saccharomyces cerevisiae AS2.504.
